SEQUENCE LISTING (2 sequences)

SEQ ID NO: 1 - DNA, 2946 bp, Homo sapiens
cgcctcccttccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtgaaccaaagaacacttgtgtggttaaagactagatataatttttccccaaactaaaatttatacttaacattggatttttaacatccaagggttaaaatacatagacattgctaaaaattggcagagcctcttctagaggctttactttctgttccgggtttgtatcattcacttggttattttaagtagtaaacttcagtttctcatgcaacttttgttgccagctatcacatgtccactagggactccagaagaagaccctacctatgcctgtgtttgcaggtgagaagttggcagtcggttagcctgggttagataaggcaaactgaacagatctaatttaggaagtcagtagaatttaataattctattattattcttaataatttttctataactatttctttttataacaatttggaaaatgtggatgtcttttatttccttgaagcaataaactaagtttcttttaaaaa

SEQ ID NO: 2 - protein, 766 aa, Homo sapiens [sequence omitted]

(19) United States
(12) Patent Application Publication
(10) Pub. No.: US 2015/0139946 A1
(43) Pub. Date: May 21, 2015

(54) DETECTING MUTATIONS IN DISEASE OVER TIME

(71) Applicant: Trovagene, Inc., San Diego, CA (US)

(72) Inventors: Mark G. Erlander, Carlsbad, CA (US); Karena Kosco, Carlsbad, CA (US); Cecile Rose Vibat, San Diego, CA (US)

(21) Appl. No.: 14/517,878

(22) Filed: Oct. 19, 2014

Related U.S. Application Data
(60) Provisional application No. 61/893,216, filed on Oct. 19, 2013.
(60) Provisional application No. 61/977,085, filed on Apr. 8, 2014.
(60) Provisional application No. 61/977,609, filed on Apr. 9, 2014.
(60) Provisional application No. 62/040,363, filed on Aug. 21, 2014.

(51) Int. Cl. C12Q 1/68; A61B 10/00; A61N 5/10

(52) U.S. Cl. CPC C12Q 1/6886; A61N 5/103; A61B 10/0045; C12Q 2600/106; C12Q 2600/156; C12Q 2600/118
USPC 424/85.4; 514/19.3; 514/152; 424/94.6; 424/141.1; 424/649; 514/64; 514/179; 424/93.71; 424/277.1; 435/6.11; 435/6.12; 506/9; 506/2

(57) ABSTRACT

Provided is a method for monitoring a gene mutation associated with a cancer in a patient over time. Also provided is a method of selecting and/or applying treatment or therapy for a subject.

### CROSS-REFERENCE TO RELATED APPLICATIONS

This application claims the benefit of U.S. Provisional Application No. 61/893,216, filed Oct. 19, 2013, U.S. Provisional Application No. 61/977,085, filed Apr. 8, 2014, U.S. Provisional Application No. 61/977,609, filed Apr. 9, 2014, and U.S. Provisional Application No. 62,040,363, filed Aug. 21, 2014, all of which are incorporated by reference herein in their entirety.

### BACKGROUND OF THE INVENTION

(1) Field of the Invention

The present invention generally relates to cancer mutations. More specifically, the invention provides methods for monitoring cancer mutations over time, which is useful for evaluating treatment options.

(2) Description of the Related Art

Nucleic acids in cancerous tissues, circulating cells, and cell-free (cf) nucleic acids present in bodily fluids can aid in identifying and selecting individuals with cancer or other diseases associated with such genetic alterations. See, e.g., Spindler et al., 2012; Benesova et al., 2013; Dawson et al., 2013; Forshew et al., 2012; Shaw et al., 2012. Some data suggest that the amount of mutant DNA in blood correlates with tumor burden and can be used to identify the emergence of resistant mutations (Forshew et al., 2012; Murtaza et al., 2013; Dawson et al., 2013; Diaz et al., 2012; Misale et al., 2012; Diehl et al., 2008). However, it is unknown whether quantitative or semi-quantitative measurements of cfDNA in blood or urine reflect tumor burden accurately enough to utilize in making treatment decisions.

There is a need for additional non-invasive methods of determining effectiveness of treatment by monitoring tumor burden over time. The present invention addresses that need.

### BRIEF SUMMARY OF THE INVENTION

The present invention is based in part on the discovery that cancer treatment can be monitored by measuring cfDNA in urine or blood at various time points over the course of the treatment.

Thus, in some embodiments, a method is provided for monitoring a gene mutation associated with a cancer in a patient over time. The method comprises

(a) obtaining a sample of a bodily fluid from the patient;

(b) quantitatively or semi-quantitatively determining the amount of the mutation in cell free DNA (cfDNA) in the sample; and

(c) repeating (a) and (b) at a later time.

Also provided is a method of selecting and/or applying treatment or therapy for a subject. The method comprises monitoring a gene mutation by the above method, and selecting and/or applying a treatment or therapy based on the detecting.

### BRIEF DESCRIPTION OF THE DRAWINGS

FIG. 1 illustrates an exemplary two-step assay design for a 28-30 bp footprint in a target gene sequence.

FIG. 2 are graphs of experimental results showing positive and negative controls for the identification of a BRAF V600E mutation.

FIG. 3 is a graph showing results of BRAF V600E monitoring of a metastatic melanoma patient before treatment, during treatment, and after treatment. No significant recurrence of disease is observed.

FIG. 4 is a graph showing results of BRAF V600E monitoring of a metastatic colorectal cancer patient before treatment, during treatment, and after treatment. Recurrence of disease is observed.

FIG. 5 is a graph showing results of BRAF V600E monitoring of a patient with appendiceal cancer before treatment and during treatment.

FIG. 6 is a graph showing results of BRAF V600E monitoring of a metastatic non-small cell lung cancer patient during treatment. Resistance to the therapy is observed.

FIG. 7 is a graph showing results of BRAF V600E monitoring of an untreated metastatic non-small cell lung cancer patient. Disease progression is observed.

FIG. 8 is a diagram of experimental results showing high concordance of KRAS status between urine, plasma and tissue samples of advanced colorectal cancer patients.

FIG. 9 is a diagram of experimental results showing the monitoring of cfDNA containing the BRAF V600E mutation in relation to response to treatment or therapy of metastatic cancer patients. ctDNA indicates “circulating tumor DNA” that is present in cfDNA.

### DETAILED DESCRIPTION OF THE INVENTION

As used herein, the singular forms “a”, “an” and “the” are intended to include the plural forms as well, unless the context clearly indicates otherwise. Additionally, the use of “or” is intended to include “and/or” unless the context clearly indicates otherwise.

As used herein, the term “sample” refers to anything which may contain an analyte for which an analyte assay is desired. In many cases, the analyte is a cf nucleic acid molecule, such as a DNA or cDNA molecule encoding all or part of BRAF. The sample may be a biological sample, such as a biological fluid or a biological tissue. Examples of biological fluids include urine, blood, plasma, serum, saliva, semen, stool, sputum, cerebrospinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

As used herein, a “patient” includes a mammal. The mammal can be e.g., any mammal, e.g., a human, primate, bird, mouse, rat, fowl, dog, cat, cow, horse, goat, camel, sheep or a pig. In many cases, the mammal is a human being.

The present invention is based in part on the discovery that gene mutations associated with cancer and other diseases can be accurately monitored by measuring cfDNA in urine or blood at various time points over the course of the treatment. The effectiveness of this discovery is shown in the Examples, where quantitative measuring of mutations in cfDNA in urine and blood at various time points of the treatment correlated with tumor burden as assessed by radiographic measurements, as well as treatment response as assessed by time-to-failure on therapy. Such measurements can be used in evaluating treatment options.

Thus, in some embodiments, a method is provided for monitoring a gene mutation in a patient over time. The method comprises

(a) obtaining a sample of a bodily fluid from the patient;

(b) quantitatively or semi-quantitatively determining the amount of the mutation in DNA in the sample; and

(c) repeating (a) and (b) at a later time.

In various embodiments, the gene mutation is associated with a cancer.

Any bodily fluid that would be expected to have DNA can be utilized in these methods. Non-limiting examples of bodily fluids include, but are not limited to, peripheral blood, serum, plasma, urine, lymph fluid, amniotic fluid, and cerebrospinal fluid. In certain particular embodiments, such as those illustrated in the Examples, the bodily fluid is serum, plasma or urine.

In some cases, the method is performed quantitatively, such that the amount of the gene alteration is quantitatively determined and may be quantitatively compared to another measurement. In other cases, the method is performed semi-quantitatively, such that the amount of the gene alteration may be determined and then compared to another measurement simply to determine a relative increase or decrease relative to each other.

These methods are not narrowly limited to any particular gene mutations in any particular cancer, since any mutation that is associated with any cancer would be expected to be accurately monitored by these methods. Nonlimiting examples of such genes are APC, BRAF, CDK4, CTNNB1, EGFR, FGFR1, FGFR2, FGFR3, HERS, PDGFR1, PDGFR2, AKT1, Estrogen Receptor, Androgen Receptor, EZH2, FLT3, HER2, IDH1, IDH2, JAK2, KIT, KRAS, c-Myc, NOTCH1, NRAS, PIK3CA, PTEN, p53, p16, or Rb1 gene. In some embodiments, the mutation is in a BRAF gene or a KRAS gene. Exemplary mutations in those genes are BRAF V600E and the KRAS mutations G12A, G12C, G12D, G12R, G12S, G12V and G13D.

An association with BRAF V600E has been reported for various human neoplasms, including melanomas (−50%) (Davies et al., 2002; Curtin et al., 2005), papillary thyroid carcinomas (−40%) (Puxeddu et al., 2004), Langherans cell histiocytosis (57%) (Badalian-Very et al., 2010) and a variety of solid tumors (at lower frequency)(Davies et al., 2002; Brose et al., 2002; Tie et al., 2011).

A member of the serine/threonine kinase RAF family, the BRAF protein is part of the RAS-RAF-MAPK signaling pathway that plays a major role in regulating cell survival, proliferation and differentiation (Keshet and Seger, 2010). BRAF mutations constitutively activate the MEK-ERK pathway, leading to enhanced cell proliferation, survival and ultimately, neoplastic transformation (Wellbrock and Hurlstone, 2010; Niault and Baccarini, 2010). All BRAF mutated hairy cell leukemia (HCL) cases carried the V600E phospho-mimetic substitution which occurs within the BRAF activation segment and markedly enhances its kinase activity in a constitutive manner (Wan et al., 2004).

In many cases, the BRAF mutation is a BRAF V600E mutation, in which a glutamic acid (Glu or E) is substituted for a Valine (Val or V) residue at position or amino acid residue 600 of SEQ ID NO:2. Alternatively, or in addition, the BRAF mutation is a substitution of an adenine (A) for a thymine (T) nucleotide at position 1860 of SEQ ID NO:1.

Homo sapiens v-raf murine sarcoma viral oncogene homolog B1, BRAF, is encoded by the following mRNA sequence (NM_{—}004333, SEQ ID NO: 1) (wherein coding sequence is bolded and the coding sequence for amino acid residue 600 is underlined and enlarged):

```
      1 cgcctccctt ccccctcccc gcccgacagc ggccgctcgg gccccggctc tcggttataa
    61 gatggcggcg ctgagcggtg gcggtggtgg cggcgcggag ccgggccagg ctctgttcaa
  121 cggggacatg gagcccgagg ccggcgccgg cgccggcgcc gcggcctctt cggctgcgga
  181 ccctgccatt ccggaggagg tgtggaatat caaacaaatg attaagttga cacaggaaca
  241 tatagaggcc ctattggaca aatttggtgg ggagcataat ccaccatcaa tatatctgga
  301 ggcctatgaa gaatacacca gcaagctaga tgcactccaa caaagagaac aacagttatt
  361 ggaatctctg gggaacggaa ctgatttttc tgtttctagc tctgcatcaa tggataccgt
  421 tacatcttct tcctcttcta gcctttcagt gctaccttca tctctttcag tttttcaaaa
  481 tcccacagat gtggcacgga gcaaccccaa gtcaccacaa aaacctatcg ttagagtctt
  541 cctgcccaac aaacagagga cagtggtacc tgcaaggtgt ggagttacag tccgagacag
  601 tctaaagaaa gcactgatga tgagaggtct aatcccagag tgctgtgctg tttacagaat
  661 tcaggatgga gagaagaaac caattggttg ggacactgat atttcctggc ttactggaga
  721 agaattgcat gtggaagtgt tggagaatgt tccacttaca acacacaact ttgtacgaaa
  781 aacgtttttc accttagcat tttgtgactt ttgtcgaaag ctgcttttcc agggtttccg
  841 ctgtcaaaca tgtggttata aatttcacca gcgttgtagt acagaagttc cactgatgtg
  901 tgttaattat gaccaacttg atttgctgtt tgtctccaag ttctttgaac accacccaat
  961 accacaggaa gaggcgtcct tagcagagac tgccctaaca tctggatcat ccccttccgc
1021 acccgcctcg gactctattg ggccccaaat tctcaccagt ccgtctcctt caaaatccat
1081 tccaattcca cagcccttcc gaccagcaga tgaagatcat cgaaatcaat ttgggcaacg
1141 agaccgatcc tcatcagctc ccaatgtgca tataaacaca atagaacctg tcaatattga
1201 tgacttgatt agagaccaag gatttcgtgg tgatggagga tcaaccacag gtttgtctgc
1261 taccccccct gcctcattac ctggctcact aactaacgtg aaagccttac agaaatctcc
1321 aggacctcag cgagaaagga agtcatcttc atcctcagaa gacaggaatc gaatgaaaac
1381 acttggtaga cgggactcga gtgatgattg ggagattcct gatgggcaga ttacagtggg
1441 acaaagaatt ggatctggat catttggaac agtctacaag ggaaagtggc atggtgatgt
1501 ggcagtgaaa atgttgaatg tgacagcacc tacacctcag cagttacaag ccttcaaaaa
1561 tgaagtagga gtactcagga aaacacgaca tgtgaatatc ctactcttca tgggctattc
1621 cacaaagcca caactggcta ttgttaccca gtggtgtgag ggctccagct tgtatcacca
1681 tctccatatc attgagacca aatttgagat gatcaaactt atagatattg cacgacagac
1741 tgcacagggc atggattact tacacgccaa gtcaatcatc cacagagacc tcaagagtaa
1801 taatatattt cttcatgaag acctcacagt aaaaataggt gattttggtc tagctacagt
1861 gaaatctcga tggagtgggt cccatcagtt tgaacagttg tctggatcca ttttgtggat
1921 ggcaccagaa gtcatcagaa tgcaagataa aaatccatac agctttcagt cagatgtata
1981 tgcatttgga attgttctgt atgaattgat gactggacag ttaccttatt caaacatcaa
2041 caacagggac cagataattt ttatggtggg acgaggatac ctgtctccag atctcagtaa
2101 ggtacggagt aactgtccaa aagccatgaa gagattaatg gcagagtgcc tcaaaaagaa
2161 aagagatgag agaccactct ttccccaaat tctcgcctct attgagctgc tggcccgctc
2221 attgccaaaa attcaccgca gtgcatcaga accctccttg aatcgggctg gtttccaaac
2281 agaggatttt agtctatatg cttgtgcttc tccaaaaaca cccatccagg cagggggata
2341 tggtgcgttt cctgtccact gaaacaaatg agtgagagag ttcaggagag tagcaacaaa
2401 aggaaaataa atgaacatat gtttgcttat atgttaaatt gaataaaata ctctcttttt
2461 ttttaaggtg aaccaaagaa cacttgtgtg gttaaagact agatataatt tttccccaaa
2521 ctaaaattta tacttaacat tggattttta acatccaagg gttaaaatac atagacattg
2581 ctaaaaattg gcagagcctc ttctagaggc tttactttct gttccgggtt tgtatcattc
2641 acttggttat tttaagtagt aaacttcagt ttctcatgca acttttgttg ccagctatca
2701 catgtccact agggactcca gaagaagacc ctacctatgc ctgtgtttgc aggtgagaag
2761 ttggcagtcg gttagcctgg gttagataag gcaaactgaa cagatctaat ttaggaagtc
2821 agtagaattt aataattcta ttattattct taataatttt tctataacta tttcttttta
2881 taacaatttg gaaaatgtgg atgtctttta tttccttgaa gcaataaact aagtttcttt
2941 taaaaa
```

Homo sapiens v-raf murine sarcoma viral oncogene homolog B1, BRAF, is encoded by the following amino acid sequence (NP_{—}004324, SEQ ID NO: 2) (wherein amino acid residue 600 is bolded and underlined and enlarged):

```
    1 maalsggggg gaepgqalfn gdmepeagag agaaassaad paipeevwni kqmikltqeh
  61 iealldkfgg ehnppsiyle ayeeytskld alqqreqqll eslgngtdfs vsssasmdtv
121 tsssssslsv lpsslsvfqn ptdvarsnpk spqkpivrvf lpnkqrtvvp arcgvtvrds
181 lkkalmmrgl ipeccavyri qdgekkpigw dtdiswltge elhvevlenv pltthnfvrk
241 tfftlafcdf crkllfqgfr cqtcgykfhq rcstevplmc vnydqldllf vskffehhpi
301 pqeeaslaet altsgsspsa pasdsigpqi ltspspsksi pipqpfrpad edhrnqfgqr
361 drsssapnvh intiepvnid dlirdqgf rg dggsttglsa tppaslpgsl tnvkalqksp
421 gpqrerksss ssedrnrmkt lgrrdssddw eipdgqitvg qrigsgsfgt vykgkwhgdv
481 avkmlnvtap tpqqlqafkn evgvlrktrh vnillfmgys tkpqlaivtq wcegsslyhh
541 lhiietkfem iklidiarqt aqgmdylhak siihrdlksn niflhedltv kigdfglatv
601 ksrwsgshqf eqlsgsilwm apevirmqdk npysf qsdvy afgivlyelm tgqlpysnin
661 nrdqiifmvg rgylspdlsk vrsncpkamk rlmaeclkkk rderplfpqi lasiellars
721 lpkihrsase pslnragfqt edfslyacas pktpigaggy gafpvh
```

Non-limiting examples of cancer include, but are not limited to, adrenal cortical cancer, anal cancer, bile duct cancer, bladder cancer, bone cancer, brain or a nervous system cancer, breast cancer, cervical cancer, colon cancer, rectal cancer, colorectal cancer, endometrial cancer, esophageal cancer, Ewing family of tumor, eye cancer, gallbladder cancer, gastrointestinal carcinoid cancer, gastrointestinal stromal cancer, Hodgkin Disease, intestinal cancer, Kaposi Sarcoma, kidney cancer, large intestine cancer, laryngeal cancer, hypopharyngeal cancer, laryngeal and hypopharyngeal cancer, leukemia, acute lymphocytic leukemia (ALL), acute myeloid leukemia (AML), chronic lymphocytic leukemia (CLL), chronic myeloid leukemia (CML), chronic myelomonocytic leukemia (CMML), non-HCL lymphoid malignancy (hairy cell variant, splenic marginal zone lymphoma (SMZL), splenic diffuse red pulp small B-cell lymphoma (SDRPSBCL), chronic lymphocytic leukemia (CLL), prolymphocytic leukemia, low grade lymphoma, systemic mastocytosis, or splenic lymphoma/leukemia unclassifiable (SLLU)), liver cancer, lung cancer, non-small cell lung cancer, small cell lung cancer, lung carcinoid tumor, lymphoma, lymphoma of the skin, malignant mesothelioma, multiple myeloma, nasal cavity cancer, paranasal sinus cancer, nasal cavity and paranasal sinus cancer, nasopharyngeal cancer, neuroblastoma, non-Hodgkin lymphoma, oral cavity cancer, oropharyngeal cancer, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcoma, adult soft tissue sarcoma, skin cancer, basal cell skin cancer, squamous cell skin cancer, basal and squamous cell skin cancer, melanoma, stomach cancer, small intestine cancer, testicular cancer, thymus cancer, thyroid cancer, uterine sarcoma, uterine cancer, vaginal cancer, vulvar cancer, Waldenstrom Macroglobulinemia, and Wilms Tumor.

Non-limiting examples of non-HCL lymphoid malignancy include, but are not limited to, hairy cell variant (HCL-v), splenic marginal zone lymphoma (SMZL), splenic diffuse red pulp small B-cell lymphoma (SDRPSBCL), splenic leukemia/lymphoma unclassifiable (SLLU), chronic lymphocytic leukemia (CLL), prolymphocytic leukemia, low grade lymphoma, systemic mastocytosis, and splenic lymphoma/leukemia unclassifiable (SLLU).

In various embodiments of the methods described herein, the patients are humans. The patients may be of any age, including, but not limited to infants, toddlers, children, minors, adults, seniors, and elderly individuals.

In any of the methods described herein, the mutation can be determined, or quantified, by any method known in the art. Nonlimiting examples include MALDI-TOF, HR-melting, di-deoxy-sequencing, single-molecule sequencing, use of probes, pyrosequencing, second generation high-throughput sequencing, SSCP, RFLP, dHPLC, CCM, or methods utilizing the polymerase chain reaction (PCR), e.g., digital PCR, quantitative-PCR, or allele-specific PCR (where the primer or probe is complementary to the variable gene sequence). In some embodiments, the PCR is droplet digital PCR, e.g., as described in the Examples. In some of these methods, the mutation is quantified along with the wildtype sequence, to determine the percentage of mutated sequence. In other methods, only the mutation is quantified.

In many embodiments, the DNA is cell free DNA (“cfDNA”). In some embodiments, the amplified or detected DNA molecule is genomic DNA. In other embodiments, the amplified or detected molecule is a cDNA.

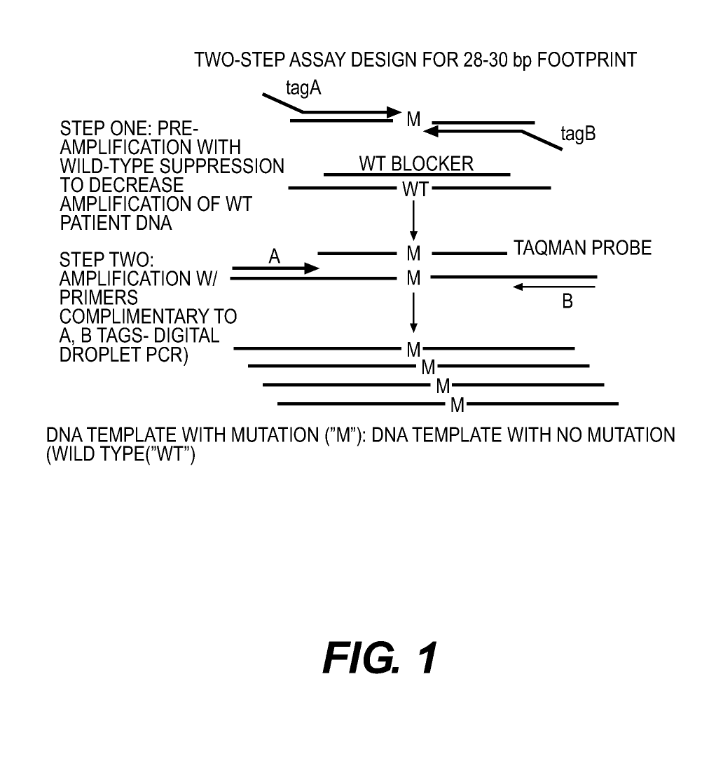

The skilled artisan can determine useful primers for PCR amplification of any mutant sequence for any of the methods described herein. In some embodiments, the PCR amplifies a sequence of less than about 50 nucleotides, e.g., as described in US Patent Application Publication US/2010/0068711. In other embodiments, the PCR is performed using a blocking oligonucleotide that suppresses amplification of a wildtype version of the gene, e.g., as illustrated in FIG. 1 (see also Example 1 below) or as described in U.S. Pat. No. 8,623,603 or U.S. Provisional Patent Application No. 62/039,905. In many embodiments, one or more primers contains an exogenous or heterologous sequence (such as an adapter or “tag” sequence), as is known in the art, such that the resulting amplified molecule has a sequence that is not naturally occurring.

The detection limits for the presence of a gene alteration (mutation) in cf nucleic acids may be determined by assessing data from one or more negative controls (e.g. from healthy control subjects or verified cell lines) and a plurality of patient samples. Optionally, the limits may be determined based in part on minimizing the percentage of false negatives as being more important than minimizing false positives. One set of non-limiting thresholds for BRAF V600E is defined as less than about 0.05% of the mutation in a sample of cf nucleic acids for a determination of no mutant present or wild-type only; the range of about 0.05% to about 0.107% as “borderline”, and greater than about 0.107% as detected mutation. In other embodiments, a no-detection designation threshold for the mutation is set at less than about 0.1%, less than about 0.15%, less than about 0.2%, less than about 0.3%, less than about 0.4%, less than about 0.5%, less than about 0.6%, less than about 0.7%, less than about 0.8%, less than about 0.9%, or less than about 1% detection of the mutation relative to a corresponding wildtype sequence.

A borderline designation can also be set according to any criteria, including the relative amount of false positives and false negatives desired.

Of course the inclusion of additional patient samples may result in the determination of different threshold values for each category, or alternatively the elimination of the “borderline” category. The desired amount of false negatives to false positives will also have an effect on the threshold value.

The “obtaining” and “determining” steps of these methods can be repeated as many times as necessary to obtain sufficient data to assist in determining treatment options or the effectiveness of the treatment being applied. In some embodiments, these steps are performed weekly, monthly, every two months, every three months, every four months, or any interval in between those time points.

In some embodiments, the patient has not previously undergone testing for the mutation in the gene. In those situations, the method are used to determine whether a specific mutation is involved in the cancer, and whether a medicament that targets the product of the gene having the mutation could be effective. For example, where a BRAF V600E mutation is present, the patient might be treated with a BRAF inhibitor such as vemurafenib, sorafenib or dabrafenib.

In some embodiments, the patient has been previously tested and a mutation determined, and the subsequent tests are to evaluate the progression of the disease and/or the effectiveness of treatment. In some cases, the detecting may identify the non-responsiveness to a treatment or therapy, and the selecting and/or applying comprises a different treatment or therapy. In other cases, the detecting may identify the responsiveness to a treatment or therapy, and the selecting and/or applying comprises continuation of the same treatment or therapy. In additional embodiments, the monitoring is a surveillance of patients, e.g., treated patients deemed “disease free” where there is a chance of recurrence.

Thus, these methods may be used to confirm the maintenance of a disclosed treatment or therapy against various diseases including cancer; or to change the treatment or therapy against the disease. In that context, a method of selecting and/or applying treatment or therapy for a subject is also provided herein. The method comprises monitoring a gene mutation by the above method, and selecting and/or applying a treatment or therapy based on the detecting.

In some embodiments of these methods, the monitoring identifies low responsiveness or non-responsiveness to a treatment or therapy, and the selecting and/or applying comprises a different treatment or therapy. In other embodiments, the monitoring identifies effective treatment or therapy, and the selecting and/or applying comprises continuing the same treatment or therapy. In additional embodiments, monitoring identifies elimination of the mutation and the selecting and/or applying comprises discontinuing treatment.

Within the scope of changing treatment or therapy, the disclosure includes increasing the treatment or therapy; reducing the treatment or therapy, optionally to the point of terminating the treatment or therapy; terminating the treatment or therapy with the start of another treatment or therapy; and adjusting the treatment or therapy as non-limiting examples. Non-limiting examples of adjusting the treatment or therapy include reducing or increasing the therapy, optionally in combination with one or more additional treatments or therapies; or maintaining the treatment or therapy while adding one or more additional treatments or therapies.

In some cases, the observation of cell-free (cf) nucleic acids identifies an increase in the levels of cf nucleic acids containing the mutation following the start of a treatment or therapy. Following the increase, the observation may reach an inflection point, where the levels decrease, or continue to increase. The presence of an inflection point may be used to determine responsiveness to the treatment or therapy, which may be maintained or reduced. A continuing decrease in the levels to be the same as, or lower than, the levels before the start of treatment of therapy is a further confirmation of responsiveness.

The absence of an inflection point indicates resistance to the treatment or therapy and so may be followed by terminating administration of the treatment or therapy, or administering at least one additional treatment or therapy against the disease or disorder to the patient, reducing the treatment of the subject with the treatment or therapy and administering at least one additional treatment or therapy against the disease or disorder to the subject.

In other cases, and following an inflection point and a decrease in levels, an additional inflection point may be observed. This may indicate the development of resistance to the treatment or therapy and be followed by terminating administration of the treatment or therapy, or administering at least one additional treatment or therapy against the disease or disorder to the subject, or reducing the treatment of the subject with the therapy and administering at least one additional therapy against the disease or disorder to the subject.

In some aspects, the monitoring of the mutation is accompanied by a determining the tumor burden, e.g., by radiography, computed tomography (CT) scanning, positron emission tomography (PET), or PET/CT scanning, and comparing the determined amount of mutation to the tumor burden. This is useful to determine whether, or confirm that the mutation being monitored is actually the driver of the tumor.

In other aspects, the determined amount of mutation is not compared to tumor burden, either at one, more than one, or all the mutation monitoring times. Given the reliability of the mutation monitoring procedures described herein, a tumor burden assessment need not be made at each time point, thus saving the patient a tumor burden assessment.

In additional aspects, the monitoring comprises evaluating a mutation that is associated with a time-to-failure parameter (i.e., the treatment directed to the mutation is known to fail after a certain period of effectiveness). In these aspects, the monitoring can assist in more accurately predicting when failure will occur, for example when the concentration of the mutation increases over a previous assessment.

Treatments and therapies of the disclosure include all modalities of cancer therapy. Non-limiting examples of these modalities include radiation therapy, chemotherapy, hormonal therapy, immunotherapy, and surgery. Non-limiting examples of radiation therapy include external beam radiation therapy, such as with photons (gamma radiation), electrons, or protons; stereotactic radiation therapy, such as with a single high dose or multiple fractionated doses to a small target; brachytherapy; and systemic radioactive isotopes.

Non-limiting examples of chemotherapy include cytotoxic drugs; antimetabolites, such as folate antagonists, purine antagonists, and pyrimidine antagonists; biological response modifiers, such as interferons; DNA damaging agents, such as bleomycin; DNA alkylating and cross-linking agents, such as nitrosourea and bendamustine; enzymatic activities, such as asparaginase; hormone antagonists, such as fulvestrant and tamoxifen; aromatase inhibitors; monoclonal antibodies; antibiotics such as mitomycin; platinum complexes such as cisplatin and carboplatin; proteasome inhibitors such as bortezomib; spindle poison such as taxanes or vincas or derivatives of either; topoisomerase I and II inhibitors, such as anthracyclines, camptothecins, and podophyllotoxins; tyrosine kinase inhibitors; anti-angiogenesis drugs; and signal transduction inhibitors.

Non-limiting examples of hormonal therapy include hormone antagonist therapy, hormone ablation, bicalutamide, enzalutamide, tamoxifen, letrozole, abiraterone, prednisone, or other glucocorticosteroid. Non-limiting examples of immunotherapy include anti-cancer vaccines and modified lymphocytes.

In some cases, the maintenance of, or change in, treatment or therapy is within one of these modalities. In other cases, the maintenance of, or change in, treatment or therapy is between two or more of these modalities. Of course a skilled clinician is aware of the recognized and approved treatments and therapies for a given disorder or disease, such as a particular cancer or tumor type, and so the maintenance of, or change in, treatment or therapy may be within those known for the disease or disorder.

The present disclosure also provides, in part, a kit for performing the disclosed methods. The kit may include a specific binding agent that selectively binds to a BRAF mutation, and instructions for carrying out the method as described herein.

One skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3rd edition), Cold Spring Harbor Press, Cold Spring Harbor, N.Y. (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, Pa., 18th edition (1990). These texts can, of course, also be referred to in making or using an aspect of the disclosure.

Preferred embodiments are described in the following examples. Other embodiments within the scope of the claims herein will be apparent to one skilled in the art from consideration of the specification or practice of the invention as disclosed herein. It is intended that the specification, together with the examples, be considered exemplary only, with the scope and spirit of the invention being indicated by the claims, which follow the examples.

### Examples

### Example 1

### Materials and Methods

The following methods were utilized in the examples that follow.

### Patient Urine Samples

A total of 27 patients with metastasized cancers, whose tumor samples were previously tested for mutations in BRAF (20 patients) and KRAS (7 patients) by a CLIA-certified laboratory, were prospectively enrolled.

Single or multiple sequential urine samples (90-110 ml or 24 hour urine collection) for cfDNA mutation analysis were obtained at baseline and during therapy and post-therapy.

### Two-Step Assay Design

A two-step assay design was developed for a 28-30 basepair footprint in the target mutant gene sequence. This assay design (and other assays known in the art) is useful for amplifying any size sequence in various tissues or bodily fluids, for example less than 400, less than 300, less than 200, less than 150 bp, less than 100 bp, less than 50 bp, less than 40 bp, less than 35 bp, or less than 30 bp.

FIG. 1 summarizes the assay design, which includes a first pre-amplification step to increase the number of copies of a target mutant gene sequence relative to wild-type gene sequences that are present in the sample. The pre-amplification is conducted in the presence of a wild-type (non-mutant) suppressing “WT blocker” oligonucleotide that is complementary to the wild-type sequence (but not the mutant sequence) to decrease amplification of wild-type DNA. The pre-amplification is performed with primers that include adapters (or “tags”) at the 5′ end to facilitate amplification in the second step.

The second step is additional amplification with primers complementary to the tags on the ends of the primers used in the first step and a TaqMan (reporter) probe oligonucleotide complementary to the mutant sequence for quantitative, digital droplet PCR.

### Assay Development

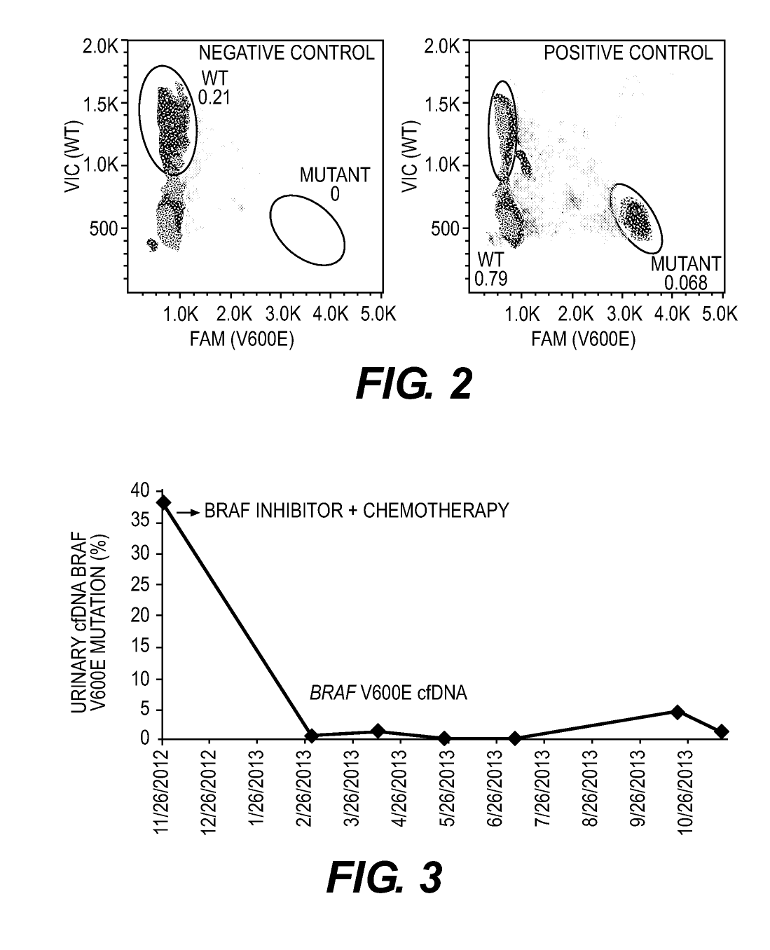

Cell lines with respective mutations (BRAF V600E, KRAS G12D, or KRAS G12V) were used as positive controls. Cell lines confirmed as wildtype BRAF and KRAS were used as negative controls. See FIG. 2.

Thresholds for mutation detection were determined by assessing data from 50 healthy controls and 39 patient samples using a classification tree. Minimizing the percentage of false negatives was given a higher importance than minimizing false positives.

A set of non-limiting thresholds for BRAF V600E were defined: <0.05% as no detection or wild-type; the range of 0.05% to 0.107% as “borderline”, and >0.107% as detected mutation. A count of KRAS G12 mutations per sample was used as a non-limiting means to confirm CLIA-identified G12 healthy (wild-type) and G12 mutation samples: <234 mutant fragments as wild-type; and 489-2825 mutant fragments as detected mutation.

### Example 2

### BRAF V600E Mutations in cfDNA

The sensitivity of the two-step assay was first assessed in urine samples from 19 patients with cancers identified as having a BRAF V600E mutation by a CLIA laboratory. The agreement rate of CLIA V600E to urinary cfDNA V600E mutation and “borderline” was 95% as shown in Table 1.

TABLE 1

| | Tumor | Urinary cfDNA BRAF |
|---|---|---|
| Tumor type and patient no. | (CLIA) | V600E mutation ( % )* |
| Non-small cell lung cancer; 15 | V600E | V600E (0.17) |
| Papillary thyroid carcinoma; 19 | V600E | V600E (0.17) |
| Non-small cell lung cancer; 16 | V600E | V600E (1.08) |
| Melanoma; 5 | V600E | V600E (37.9) |
| Non-small cell lung cancer; 13 | V600E | V600E (0.68) |
| Colorectal cancer; 1 | V600E | V600E (21.12) |
| Melanoma; 8 | V600E | V600E (0.13) |
| Colorectal cancer; 3 | V600E | V600E (1.49) |
| Glioblastoma; 19 | V600E | V600E (5.36) |
| Melanoma; 10 | V600E | Borderline |
| | | V600E (0.07) |
| Melanoma; 11 | V600E | Negative |
| | | V600E (0.04) |
| Melanoma; 9 | V600E | V600E (0.15) |
| Adenocarcinoma of unknown | V600E | Borderline |
| primary; 14 | | V600E (0.07) |
| Colorectal cancer; 2 | V600E | V600E (416.58) |
| Non-small cell lung cancer; 12 | V600E | V600E (2.93) |
| Melanoma; 7 | V600E | V600E (0.97) |
| Papillary thyroid carcinoma; 18 | V600E | V600E (1.66) |
| Melanoma; 6 | V600E | V600E (1.01) |
| Ovarian cancer; 17 | V600E | Borderline |
| | | V600E (0.08) |
| Appendiceal cancer; 4 | V600E | V600E (3.43) |

*In patients with several sequential urine collections over time, samples with highest mutant fraction are indicated.

Further concordance of the presence of a BRAF V600E mutation in tissue (by a CLIA laboratory) to urinary cfDNA V600E mutation was observed with both baseline urine samples (before treatment) and any assessed point of urine sample. Those results are provided in Table 2 and 3.

TABLE 2
Concordance of BRAF V600E Tissue (CLIA) to Baseline Urine cfDNA

| Tested (N = 33) | BRAF Mutation Urine | BRAF Wild Type Urine |
|---|---|---|
| BRAF Mutation CLIA | 25 | 7 |
| BRAF Wild Type CLIA | 0 | 0 |

| | |
|---|---|
| Observed Agreements | 25 (76%) |

TABLE 3
Concordance of BRAF V600E Tissue (CLIA) to Any Assessed Point of
Urine cfDNA

| Tested (N − 33) | BRAF Mutation Urine | BRAF Wild Type Urine |
|---|---|---|
| BRAF Mutation CLIA | 31 | 2 |
| BRAF Wild Type CLIA | 0 | 0 |

| | |
|---|---|
| Observed Agreements | 31 (94%) |

Additionally, cfDNA with the BRAF V600E mutation correlates with its presence in tissue samples from advanced cancer patients, as shown in Table 4. The BRAF V600E mutation was detected in the urine of patients with colorectal, NSCLC (non-small cell lung cancer), ovarian, melanoma, papillary thyroid cancers and other cancers. The disclosed V600E assay demonstrated high concordance in comparison to tissue biopsies (88% detected in urine at any time point tested; 29 of 33 subjects).

TABLE 4

| | | Baseline | Longitudinal |
|---|---|---|---|
| | | Urinary | Urinary |
| | | BRAF V600E | BRAF V600E |
| | | cfDNA | cfDNA |
| Tumor Type | Tissue (CLIA) | Detection | Detection |
| Appendiceal | BRAF V600E | Mutant | Mutant |
| Adenocarcinoma | BRAF V600E | Mutant | Mutant |
| Cholangiocarcinoma | BRAF V600E | Mutant | Mutant |
| Colorectal Cancer | BRAF V600E | Mutant | Mutant |
| Colorectal Cancer | BRAF V600E | Mutant | Mutant |
| Melanoma | BRAF V600E | Mutant | Mutant |
| NSCLC | BRAF V600E | Low Mutant | Mutant |
| NSCLC | BRAF V600E | Mutant | Mutant |
| Papillary Thyroid | BRAF V600E | Low Mutant | Mutant |
| Papillary Thyroid | BRAF V600E | Mutant | Not Done |

### Example 3

### KRAS G12D Mutations in cfDNA

The sensitivity of the two-step assay was also assessed in urine samples from 7 patients with cancers identified as having a KRAS G12D mutation by a CLIA laboratory. The agreement rate of CLIA G12D to urinary cfDNA G12D mutation was 100% as shown in Table 5.

| | | Baseline G12 KRAS- |
|---|---|---|
| | Tumor | mutant urinary cfDNA |
| Tumor Type | (CLIA) | (mutant fragments) |
| Colorectal Cancer | G12D | G12D (489) |
| Colorectal Cancer | G12D | G12D (563) |
| Colorectal Cancer | G12D | G12D (1935) |
| Colorectal Cancer | G12D | G12D (2825) |
| Colorectal Cancer | G12V | G12D (1168) |
| Non-Small Cell Lung Cancer | G12V | G12D (1083) |
| Appendiceal Cancer | G12D | G12D (1231) |

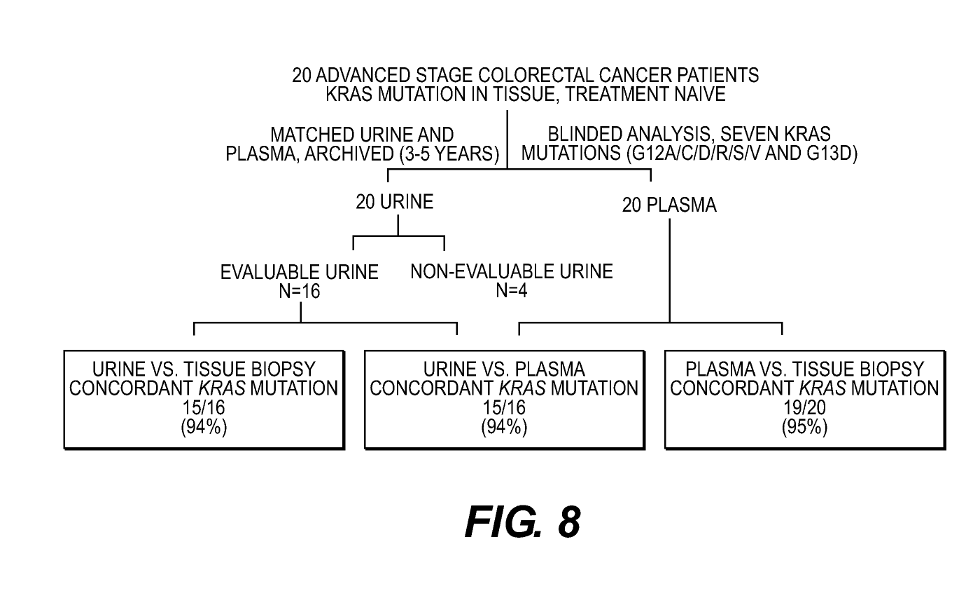

Matched urine and plasma samples that had been archived 3-5 years from 20 advanced stage and treatment naïve colorectal cancer patients were assessed as described herein for the KRAS mutation in comparison to matched tissue samples. The results are shown in FIG. 8, which illustrates the high concordance between all three sample types.

### Example 3

### Longitudinal Assessment of cfDNA Mutations

In three patients a series of multiple urine samples obtained over time was assayed as described above. The patients were afflicted with metastatic melanoma (treated with a BRAF inhibitor and chemotherapy), metastatic colorectal cancer (treated with a BRAF inhibitor and an anti-EGFR antibody), and appendiceal cancer (treated with a BRAF inhibitor and a kinase inhibitor).

The results for the melanoma patient are shown in FIG. 3. A signal of 37.9% was observed in the patient's initial sample, followed by the start of therapy. The subsequent four samples had values of 0.08%, 0.83%, 0.17%, and 0.04%. After termination of treatment, the observed levels of the BRAF V600E mutation in urinary cfDNA remained low.

The results for the colorectal cancer patient are shown in FIG. 4. A signal of 1.49% was observed in the patient's initial sample, followed by the start of therapy. The subsequent four samples had values of 0.09%, 0.00%, 0.00%, and 0.00%. After termination of treatment, the observed levels of the BRAF V600E mutation in urinary cfDNA remained low and then began to increase.

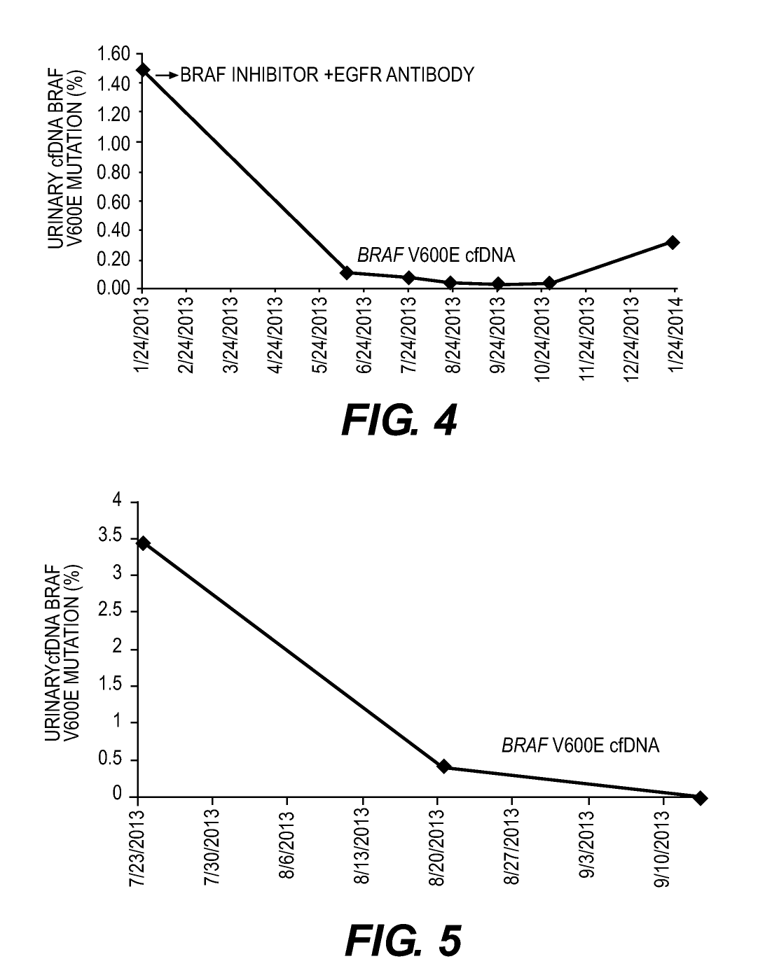

The results for the appendiceal patient are shown in FIG. 5. A signal of 3.43% was observed in the patient's initial sample which was concurrent with therapy. The subsequent two samples had values of 0.45% and 0.02%.

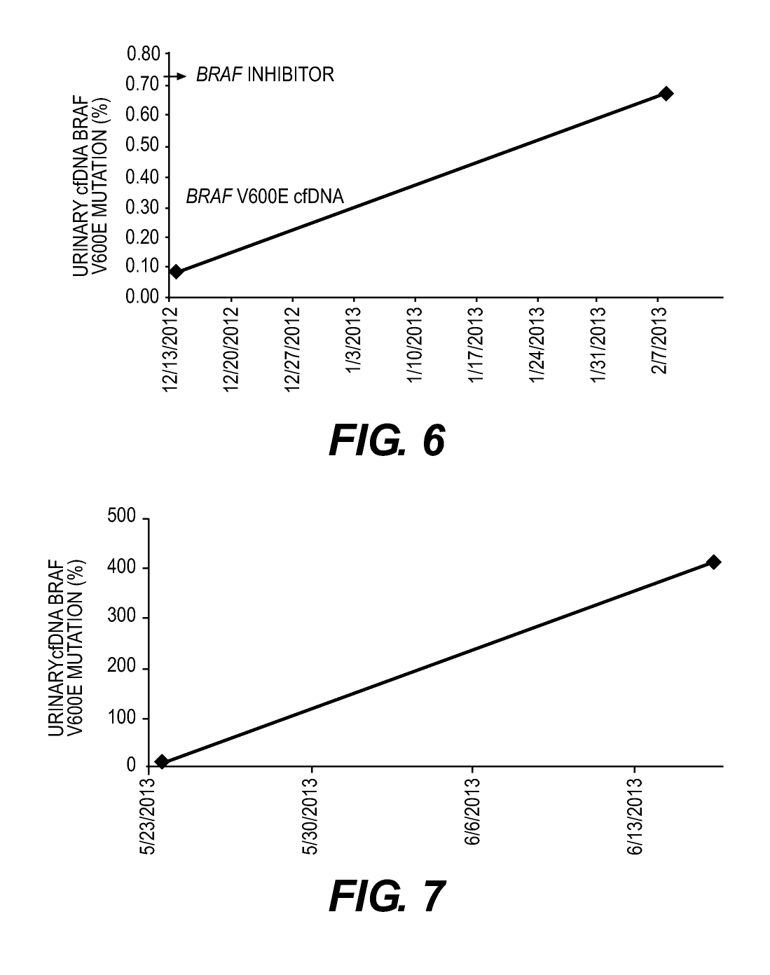

In a fourth and fifth patients with metastatic non-small cell lung cancer, resistance to a BRAF inhibitor was observed during treatment of one patient (FIG. 6). The increase in BRAF V600E mutation in urinary cfDNA urinary was similar to that of an untreated patient (FIG. 7).

In total, longitudinal analysis of BRAF V600E in 17 of 32 metastatic cancer patients was performed by testing serially collected urine. The dynamics of urinary cell-free BRAF V600E correlated with responsiveness (or lack of response) to therapy in 13 of 17 advanced cancer patients (76%).

### Example 4

### Monitoring Presence of BRAF V600E Mutation Vs. Treatment Response

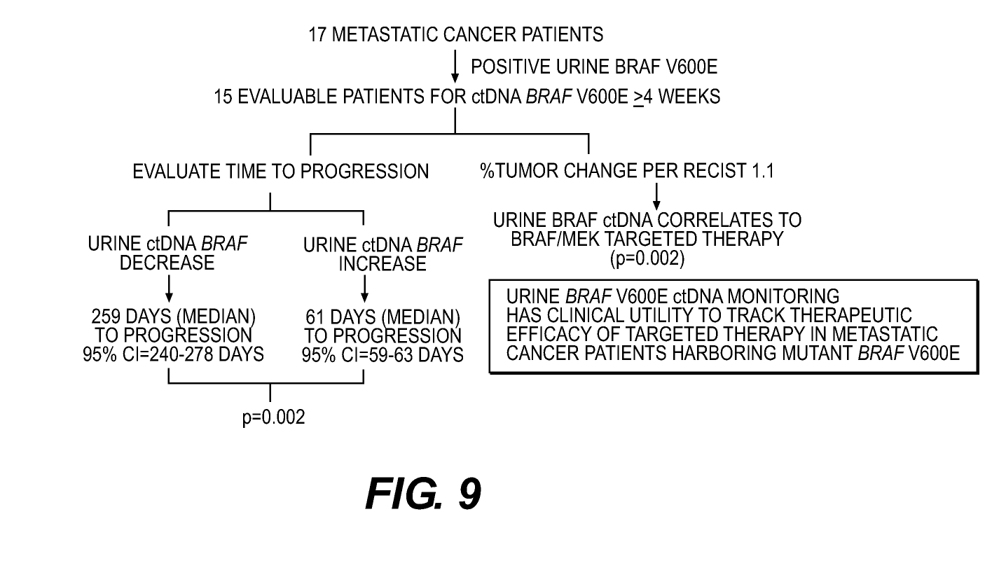

In 15 of 17 metastatic cancer patients that were positive for BRAF V600E cfDNA in urine, the BRAF V600E cfDNA (or ctDNA, circulating tumor DNA) in urine was evaluated over time to monitor disease progression and/or responsiveness to therapy. As shown in FIG. 9, the monitoring has clinical utility for tracking the therapeutic efficacy of targeted therapy in metastatic cancer patients with detectable BRAF V600E cfDNA or ctDNA.

### REFERENCES

Badalian-Very et al., 2010, Blood 116:1919-23.
Benesova et al., 2013, Anal Biochem. 433:227-34.
Brose et al., 2002, Cancer Res 62:6997-7000.
Curtin et al., 2005, N Engl J Med 353:2135-47.
Davies et al., 2002, Nature 417:949-54.
Dawson et al., 2013, N Engl J Med. 368:1199-1209.
Diehl et al., 2008, Nat Med. 14:985-990.
Forshew et al., 2012, Science Translational Medicine, 4:136ra168.
Keshet Y and Seger R., 2010, Methods Mol Biol. 661:3-38.
Niault T and Baccarini M., 2010, Carcinogenesis. 31:1165-74.
Puxeddu et al., 2004, J Clin Endocrinol Metab 89:2414-20.
Shaw et al., 2012, Genome Res. 22:220-31.
Tie et al., 2011, Int J Cancer 128:2075-84.
Wan et al., 2004, Cell 116:855-67.
Wellbrock C and Hurlstone A, 2010, Pharmacol. 80:561-7.
U.S. Pat. No. 8,623,603.
US Patent Application Publication US2010/0068711.
U.S. Provisional Patent Application No. 62/039,905.

In view of the above, it will be seen that several objectives of the invention are achieved and other advantages attained.

As various changes could be made in the above methods and compositions without departing from the scope of the invention, it is intended that all matter contained in the above description and shown in the accompanying drawings shall be interpreted as illustrative and not in a limiting sense.

All references cited in this specification are hereby incorporated by reference. The discussion of the references herein is intended merely to summarize the assertions made by the authors and no admission is made that any reference constitutes prior art. Applicants reserve the right to challenge the accuracy and pertinence of the cited references.

What is claimed is:

1. A method of monitoring a gene mutation associated with a cancer in a patient over time, the method comprising
(a) obtaining a sample of a bodily fluid from the patient;
(b) quantitatively or semi-quantitatively determining the amount of the mutation in cell free DNA (cfDNA) in the sample; and
(c) repeating (a) and (b) at a later time.

2. The method of claim 1, wherein the bodily fluid is serum or plasma.

3. The method of claim 1, wherein the bodily fluid is urine.

4. The method of claim 1, wherein the mutation is in a APC, BRAF, CDK4, CTNNB1, EGFR, FGFR1, FGFR2, FGFR3, HERS, PDGFR1, PDGFR2, AKT1, Estrogen Receptor, Androgen Receptor, EZH2, FLT3, HER2, IDH1, IDH2, JAK2, KIT, KRAS, c-Myc, NOTCH1, NRAS, PIK3CA, PTEN, p53, p16, or Rb1 gene.

5. The method of claim 1, wherein the mutation is BRAF V600E or KRAS mutations G12A, G12C, G12D, G12R, G12S, G12V or G13D.

6. The method of claim 1, wherein the testing comprises sequencing.

7. The method of claim 1, wherein the testing comprises polymerase chain reaction (PCR).

8. The method of claim 7, wherein the PCR is droplet digital PCR.

9. The method of claim 7, wherein the PCR amplifies a sequence of less than about 50 nucleotides.

10. The method of claim 7, wherein the PCR is performed using a blocking oligonucleotide that suppresses amplification of a wildtype version of the gene.

11. The method of claim 1, wherein a no-detection designation threshold for the mutation is established by examining body fluid samples from healthy subjects or diseased subjects with the wildtype status of the target gene.

12. The method of claim 1, wherein (a) and (b) are repeated at least twice.

13. The method of claim 1, wherein the patient has not previously undergone testing for the mutation.

14. The method of claim 1, wherein the patient is undergoing treatment with a medicament that targets the product of the gene having the mutation.

15. The method of claim 1, wherein the patient is undergoing treatment with a medicament that does not target the product of the gene having the mutation.

16. The method of claim 1, further comprising comparing the determined amount of mutation to tumor burden.

17. The method of claim 1, where the determined amount of mutation is not compared to tumor burden at at least one of the times that the mutation is monitored.

18. The method of claim 16, wherein the tumor burden assessment is by radiography, computed tomography (CT) scanning, positron emission tomography (PET), or PET/CT scanning.

19. A method of selecting and/or applying treatment or therapy for a subject, the method comprising monitoring a gene mutation according to claim 1, and selecting and/or applying a treatment or therapy based on the detecting.
